(19) United States
(12) Patent Application Publication
(10) Pub. No.: US 2012/0164082 A1
(43) Pub. Date: Jun. 28, 2012

(54) METHOD OF USING APPARATUS FOR PREVENTING PLAQUE, PREVENTING CATIVIES AND KEEPING TEETH CLEAN

(76) Inventors: Grace Yum Zimmermann, Chicago, IL (US); Kent Zimmermann, Chicago, IL (US)

(21) Appl. No.: 13/333,917

(22) Filed: Dec. 21, 2011

Related U.S. Application Data
(60) Provisional application No. 61/426,861, filed on Dec. 23, 2010.

(51) Int. Cl. A61K 8/34; A61Q 11/00; A61K 8/36

(52) U.S. Cl. 424/49

(57) ABSTRACT

A method for using an apparatus to gently clean an adult's or a child's teeth, gums, mouth, cheek surfaces and tongue with a liquid-filled cotton swab containing a proprietary solution that uses a natural sugar, in this case xylitol, that may prevent plaque from sticking to the user's teeth. Application of the xylitol solution may also kill bacteria and help prevent cavities from forming. The swab may be particularly useful for people that may have difficulty in brushing or flossing, such as victims of stroke or paralysis, people who cannot fully open their mouths, wearers of orthodontia, children, and babies.

### CROSS-REFERENCE TO RELATED APPLICATIONS

The present application claims the benefit of priority of U.S. provisional application No. 61/426,861, filed Dec. 23, 2010, the contents of which are herein incorporated by reference.

### BACKGROUND OF THE INVENTION

The present invention relates to oral hygiene products and methods and, more particularly, to a liquid filled swab that may be applied to a user's teeth to prevent plaque from sticking to the teeth, to kill bacteria, and to prevent cavities.

Children can start getting cavities as soon as they get teeth, and more than one in four children now get a cavity before they enter kindergarten. Childhood cavities are the single most prevalent chronic childhood disease in the United States, and cavities in baby teeth can cause damage to permanent teeth in some cases. However, many young children, especially babies and toddlers that are just developing teeth, are not able to reliably brush and/or floss their teeth. Dentists have often advised parents to wipe their young children's teeth down with a wet cloth to remove sugar from the teeth to help prevent cavities from forming.

Some people are unable to easily brush their teeth and/or swallow due to disability, such as victims of strokes or paralysis. These people need an alternative way to maintain their oral health and to prevent plaque from sticking to their teeth.

As can be seen, there is a need for apparatus and methods to improve the oral hygiene of children and adults who may not easily be able to brush their teeth.

### SUMMARY OF THE INVENTION

In one aspect of the present invention, a oral hygiene solution comprises xylitol, in an aqueous solution comprising from about 25 to about 35% w/w xylitol; and an orally acceptable diluent or adjuvant.

In another aspect of the present invention, a oral hygiene method comprises applying a xylitol solution to at least one of a user's teeth, gums, mouth and tongue, wherein the xylitol solution is an aqueous solution comprising from about 25 to about 35% w/w xylitol.

In a further aspect of the present invention, a method for preventing plaque build-up on a user's teeth comprises applying a xylitol solution to at least one of a user's teeth, gums, mouth and tongue, wherein the xylitol solution is an aqueous solution comprising from about 25 to about 35% w/w xylitol.

These and other features, aspects and advantages of the present invention will become better understood with reference to the following drawings, description and claims.

### BRIEF DESCRIPTION OF THE DRAWINGS

FIG. 1 is a perspective view of an oral hygiene swab, in use, according to an exemplary embodiment of the present invention;

FIG. 2 is a perspective view of the oral hygiene swab of FIG. 1 being prepared for use;

FIG. 3 is a perspective view of the oral hygiene swab of FIG. 1; and

FIG. 4 is a flow chart describing a method for making and using an oral hygiene swab according to an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a method for using an apparatus for gently cleaning an adult's or a child's teeth, gums, mouth, cheek surfaces and tongue using a liquid-filled cotton swab containing a proprietary solution that uses a natural sugar, in this case xylitol, that may prevent plaque from sticking to the user's teeth. Application of the xylitol solution may also kill bacteria and help prevent cavities from forming. The swab may be particularly useful for people who may have difficulty in brushing or flossing, such as victims of stroke or paralysis, people who cannot fully open their mouths, wearers of orthodontia, children, and babies.

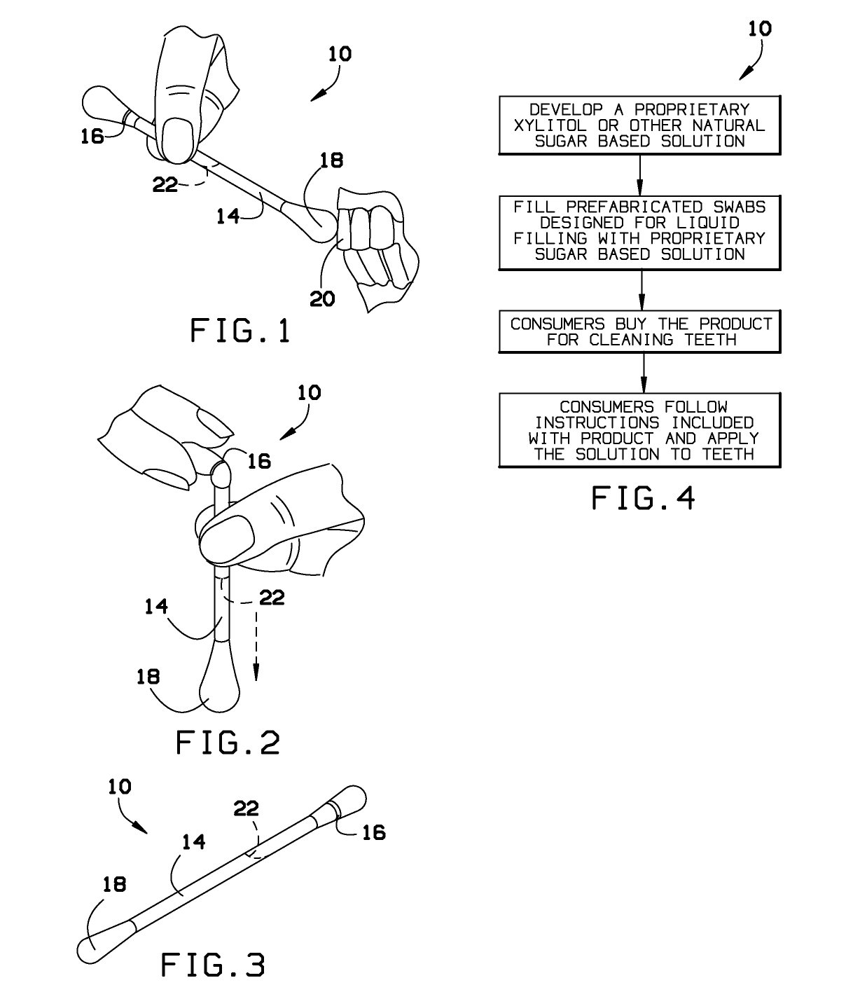

Referring now to FIGS. 1 through 4, an oral hygiene swab 10 may include a xylitol solution 22 used in conjunction with a cotton swab 12 that has a hollow center tube or stick 14 that is pre-filled with the proprietary xylitol solution 22 or another solution to maintain good oral health. The design of the pre-made and pre-filled swab prevents the xylitol solution from immediately soaking the swab until the user is ready to apply the xylitol solution to a child or other person's teeth.

According to the method of the present invention, a user first holds the xylitol solution filled swab 10 vertically with a snap end indicator ring 16 facing up. The user then snaps the tip of the swab having the snap end indicator ring 16. The xylitol solution 22 inside the tube 14 may then flow down towards the downward facing tip 18. Once the swab tip 18 is coated with the solution, the user may gently cleanse the inside of a person's mouth, gums, teeth, cheek surfaces, and tongue. The swab should then be discarded.

This apparatus and methods of the present invention may be used by one person on another's teeth, or may be used on one's own teeth, to supplement good oral hygiene.

The xylitol solution used in the present invention may be comprised of, for example, approximately 20-50%, typically from about 25-35%, xylitol and each use may deliver between about 0.75 and about 1.5 grams of xylitol. The xylitol solution may contain various other orally acceptable ingredients, diluents and adjuvants, such as thickeners, wetting agents, preservatives, flavors, and the like. The term “orally acceptable” is used herein to describe ingredients that may be approved or acceptable for use in an oral hygiene product.

In one embodiment of the present invention, the xylitol solution may contain from about 30% xylitol; about 10% glycerin; less than one percent of sodium citrate, sodium benzoate, citric acid, and flavoring; with the remainder of the formulation being water. The table below shows one exemplary formulation of the xylitol solution. Other formulations are contemplated within the scope of the present invention.

| Ingredient | % w/w |
|---|---|
| Water | 58.75 |
| Xylitol | 30.00 |
| Glycerin | 10.00 |
| Sodium citrate | 0.30 |
| Sodium benzoate | 0.20 |
| Citric acid | 0.05 |
| Natural flavor | 0.70 |

While the above description describes the application of the xylitol solution via a swab, other application methods may be within the scope of the present invention. For example, the xylitol solution described above may be distributed in a different application or in a packet to add to a drink, such as water.

It should be understood, of course, that the foregoing relates to exemplary embodiments of the invention and that modifications may be made without departing from the spirit and scope of the invention as set forth in the following claims.

What is claimed is:

1. An oral hygiene solution comprising:
xylitol, in an aqueous solution comprising from about 25 to about 35% w/w xylitol; and
an orally acceptable diluent or adjuvant.

2. The oral hygiene solution of claim 1, wherein the xylitol is present in a concentration of about 30% w/w.

3. The oral hygiene solution of claim 1, further comprising about 10% w/w glycerin.

4. The oral hygiene solution of claim 3, further comprising less than one percent of sodium citrate, sodium benzoate, citric acid and a flavor.

5. An oral hygiene method comprising:
applying a xylitol solution to at least one of a user's teeth, gums, mouth and tongue, wherein the xylitol solution is an aqueous solution comprising from about 25 to about 35% w/w xylitol.

6. The method of claim 5, wherein the xylitol solution is applied with a swab that is liquid filled with the xylitol solution.

7. The method of claim 5, further comprising preventing build-up of plaque, preventing cavities, and killing bacteria through the application of the xylitol solution.

8. The method of claim 5, wherein the xylitol is present in a concentration of about 30% w/w.

9. A method for preventing plaque build-up on a user's teeth, comprising:
applying a xylitol solution to at least one of a user's teeth, gums, mouth and tongue, wherein the xylitol solution is an aqueous solution comprising from about 25 to about 35% w/w xylitol.

10. The method of claim 9, wherein the xylitol solution is applied with a swab that is liquid-filled with the xylitol solution.
